(19) United States
(12) Patent Application Publication
(10) Pub. No.: US 2019/0000908 A1
(43) Pub. Date: Jan. 3, 2019

(54) COMPOSITIONS AND METHODS FOR TREATING AND/OR PREVENTING A URINARY TRACT INFECTION

(71) Applicant: HMS LABORATORIES LTD., Herzliya (IL)

(72) Inventor: Meir AMIR, Haifa (IL)

(21) Appl. No.: 16/010,719

(22) Filed: Jun. 18, 2018

Related U.S. Application Data
(60) Provisional application No. 62/526,453, filed on Jun. 29, 2017.

(51) Int. Cl. A61K 36/756; A61K 36/45; A61K 36/54; A61K 9/48; A61K 45/06; A61P 31/04; A61P 31/10

(52) U.S. Cl. CPC A61K 36/756; A61K 36/45; A61K 36/54; A61P 31/10; A61K 45/06; A61P 31/04; A61K 9/4858

(57) ABSTRACT

A composition comprising D-Mannose, Phellodendron Extract and Cranberry is disclosed. The disclosed composition, is useful in treating and preventing a microbial infection and induced red blood cells agglutination.

### CROSS-REFERENCE TO RELATED APPLICATIONS

This application claims the benefit of U.S. Provisional Patent Application No. 62/526,453, filed Jun. 29, 2017, and entitled “COMPOSITIONS AND METHODS FOR TREATING AND/OR PREVENTING A URINARY TRACT INFECTION”, the content of which is incorporated herein by reference in its entirety.

### INTRODUCTION

A urinary tract infection (UTI) is an infection in any part of the urinary system—kidneys, ureters, bladder and urethra. Most infections involve the lower urinary tract—the bladder and the urethra. A severe systemic reaction may occur if a UTI spreads to the kidneys. Women are at a greater risk of developing a UTI than men are, due to their urethra anatomy. Escherichia coli bacteria was found as causing of 75-95% of infections in women. This bacterium can attach to the urinary tract walls and form a biofilm that resists the body's immune response. Other rear infection source are other bacterial species and fungi.

UTIs evoke the following symptoms: a burning feeling upon urination; a frequent or intense urge to urinate, even though a small amount of urine is secreted; a pain or pressure in the back or lower abdomen; a cloudy, dark, bloody, or strange-smelling urine; feeling tired or shaky and a fever or chills.

UTI affects about 150 million people each year. UTIs are the most frequent bacterial infection in women. They occur most frequently between the ages of 16 and 35 years, with 10% of women getting an infection yearly and more than 40-60% having an infection at some point in their lives. Recurrences are common, with nearly half of people getting a second infection within a year.

The most common treatment for UTIs are antibiotics, however, increasing antibiotic resistance is causing concern about the future of treating those with complicated and recurrent UTIs.

### SUMMARY OF THE INVENTION

In some embodiments, the present invention provides a composition comprising D-Mannose and Phellodendron Extract, wherein said D-Mannose weight is at least 3.5% from the total weight of said composition.

In some embodiments, the present invention provides a composition comprising D-Mannose and Phellodendron Extract and Cranberry.

In some embodiments, the present invention provides a composition comprising D-Mannose and Phellodendron Extract, Cranberry, Methylsulfonylmethane (MSM) and Cinnamon, or any combination thereof.

In some embodiments, the present invention provides that the weight ratio of D-Mannose to Phellodendron Extract to Cranberry is from 4:4:1 to 4:1:2. In some embodiments, the present invention provides that the composition having an anti-urinary tract infection effect.

In some embodiments, the present invention provides a method of treating a subject afflicted with a urinary tract microbial infection, comprising the step of administering to said subject a therapeutically effective amount of a composition comprising: D-Mannose, Phellodendron Extract and Cranberry, thereby treating a subject afflicted with a urinary tract microbial infection.

In some embodiments, the present invention provides a method of preventing a urinary tract microbial infection in a subject, comprising the step of administering to said subject a therapeutically effective amount of a composition comprising: D-Mannose, Phellodendron Extract and Cranberry, thereby preventing a urinary tract microbial infection in a subject.

In some embodiments, the present invention provides a method for reducing a minimal effective dose of a urinary tract antibiotic in treating a subject afflicted with a urinary tract microbial infection, comprising the step of administering to said subject a therapeutically effective amount of a composition comprising: D-Mannose, Phellodendron Extract and Cranberry along with said antibiotic, thereby reducing the minimal effective dose of a urinary tract antibiotic in treating a subject afflicted with a urinary tract microbial infection.

### BRIEF DESCRIPTION OF THE DRAWING

FIGS. 1A-1B Present a Human Red Blood Cell (HRBC) agglutination assay. Test plate in natural image (FIG. 1A) and in negative image (FIG. 1B) modes. Raw A: “Negative” control (PBS); Raw B: “Positive” control [Columns 1-6 E. coli 4169 and Columns 7-12 E. coli 9775 (Mannose sensitive)]; Raw C: Column 2, 3, 5, 6 Mannose+E. coli 4169 (Mannose resistance); Columns 8,9,11,12 Mannose+E. coli 9775 (Mannose resistance); Column 1,4 E. coli 4169+PBS; Column 7,10 E. coli 9775+PBS.

FIGS. 2A-2B Present a Human Red Blood Cell (HRBC) agglutination assay. Test plate of the examined following ingredients: D-Mannose, Phellodendron Extract and Cranberry, and the exemplary formulation components (Table 1) in natural (FIG. 2A) and in negative (FIG. 2B) image modes. Raw A: “Negative” control (PBS); Raw B: Column 1,2,4,5 “Positive” control—E. coli 4169 and Columns 7,8,10,11 “Positive” control—E. coli 9775; Raw C, Column 1,2,4,5 Mannose+Phellodendron+E. coli 4169 and Columns 7,8,10,11 Mannose+Phellodendron E. coli 9775 (Columns 1,2 5 mg/ml; 4,5 1 mg/ml; 7,8 5 mg/ml; 10,11 1 mg/ml); Raw D, Column 1,2,4,5 Mannose+Cranberries+E. coli 4169 and Columns 7,8,10,11 Mannose+Cranberries+E. coli 9775 (Columns 1,2 5 mg/ml; 4,5 1 mg/ml; 7,8 5 mg/ml; 10,11 1 mg/ml); Raw E, Column 1,2,4,5 Phellodendron+Cranberries+E. coli 4169 and Columns 7,8,10,11 Phellodendron+Cranberries+E.coli 9775 (Columns 1,2 5 mg/ml; 4,5 1 mg/ml; 7,8 5 mg/ml; 10,11 1 mg/ml); Raw F, Column 1,2,4,5 Mannose+Phellodendron+Cranberries+E. coli 4169 and Columns 7,8,10,11 Mannose+Phellodendron+Cranberries+E. coli 9775 (Columns 1,2 5 mg/ml; 4,5 1 mg/ml; 7,8 5 mg/ml; 10,11 1 mg/ml); Raw G: Columns 2,3,5,6 Exemplary formulation components (Table 1)+E. coli 4169 and Columns 8,9,11,12 Exemplary formulation components (Table 1)+E. coli 9775, Columns 1,4 E. coli 4169+PBS and column 7,10 E. coli 9775+PBS. Wells G8, G9, G11 and G12 where tested and found to be another agglutination form. Raw H: Column 1,2,4,5 “Positive” control—E. coli 4169 and Columns 7,8,10,11 “Positive” control—E. coli 9775.

### DETAILED DESCRIPTION OF THE INVENTION

According to some embodiments, there is provided a composition comprising D-Mannose and Phellodendron Extract. In some embodiments, the composition comprises D-Mannose at a weight content of at least 3.5% from the total weight of the composition. In some embodiments, D-Mannose weight content is in the range of 3.5% and 90% from the total weight of the composition. In some embodiments, D-Mannose weight content is in the range of 10% and 70% from the total weight of the composition. In some embodiments, D-Mannose weight content is in the range of 30% and 60% from the total weight of the composition. In some embodiments, the composition comprises D-Mannose at a weight content of 3.5% to 90% from the total weight of the composition. In some embodiments, the composition comprises D-Mannose at a weight content of 5% to 85% from the total weight of the composition. In some embodiments, the composition comprises D-Mannose at a weight content of 5% to 50% from the total weight of the composition. In some embodiments, the composition comprises D-Mannose at a weight content of 15% to 60% from the total weight of the composition. In some embodiments, the composition comprises D-Mannose at a weight content of 15% to 75% from the total weight of the composition. In some embodiments, the composition comprises D-Mannose at a weight content of 25% to 70% from the total weight of the composition. In some embodiments, the composition comprises D-Mannose at a weight content of 30% to 60% from the total weight of the composition.

In some embodiments, the D-Mannose to Phellodendron Extract weight ratio is in the range of 28:1 and 1:4. In some embodiments, the D-Mannose to Phellodendron Extract weight ratio is in the range of 15:1 and 1:2. In some embodiments, the D-Mannose to Phellodendron Extract weight ratio is in the range of 10:1 and 1:4. In some embodiments, the D-Mannose to Phellodendron Extract weight ratio is in the range of 5:1 and 1:4. In some embodiments, the D-Mannose to Phellodendron Extract weight ratio is in the range of 5:1 and 1:2.

In some embodiments, D-mannose is of a natural source. In some embodiments, D-mannose is of a plant source. In some embodiments, D-mannose is of a fruit source. In some embodiments, D-mannose is synthetic.

In some embodiments, a Phellodendron Extract is produced from the bark part of the plant. In some embodiments, the Phellodendron Extract source is selected from the species of: Phellodendron Chinese, Phellodendron amurense, Phellodendron japonicum, Phellodendron shachalinese or Phellodendron sinii. In some embodiments, the Phellodendron Extract source is Phellodendron Chinese.

In some embodiments, Phellodendron weight content is in the range of 1 and 99% from the total weight of the composition. In some embodiments, Phellodendron weight content is in the range of 5 and 90% from the total weight of the composition. In some embodiments, Phellodendron weight content is in the range of 20 and 80% from the total weight of the composition. In some embodiments, Phellodendron weight content is in the range of 30 and 70% from the total weight of the composition. In some embodiments, Phellodendron weight content is in the range of 20 and 50% from the total weight of the composition. In some embodiments, Phellodendron weight content is in the range of 20 and 40% from the total weight of the composition.

The term “Phellodendron” refers to a plant, a genus of deciduous trees in the family Rutaceae. The bark extract is used in herbal medicine. In some embodiments, the term “Phellodendron” comprises a Phellodendron Extract. The term “Phellodendron chinense” refers to a plant species in the genus Phellodendron.

In some embodiments, the composition further comprises a Cranberry. In some embodiments, Cranberry is a Cranberry fruit extract. In some embodiments, Cranberry is a Cranberry fruit concentrate. In some embodiments, Cranberry concentrate includes but not limited to: CranMax® (Proprietary Nutritionals, Inc., NJ).

In some embodiments, D-Mannose to Cranberry weight ratio is in the range of 20:1 and 1:4. In some embodiments, D-Mannose to Cranberry weight ratio is in the range of 10:1 and 1:2. In some embodiments, D-Mannose to Cranberry weight ratio is in the range of 5:1 and 1:1. In some embodiments, Phellodendron Extract to Cranberry weight ratio is in the range of 8:1 and 1:2. In some embodiments, Phellodendron Extract to Cranberry weight ratio is in the range of 4:1 and 1:1.

In some embodiments, Cranberry weight content is in the range of 1 and 90% from the total weight of the composition. In some embodiments, Cranberry weight content is in the range of 3 and 80% from the total weight of the composition. In some embodiments, Cranberry weight content is in the range of 5 and 70% from the total weight of the composition. In some embodiments, Cranberry weight content is in the range of 10 and 50% from the total weight of the composition. In some embodiments, Cranberry weight content is in the range of 10 and 30% from the total weight of the composition. In some embodiments, Cranberry weight content is in the range of 10 and 20% from the total weight of the composition.

In some embodiments, the composition further comprises Methylsulfonylmethane (MSM). In some embodiments, MSM is of a natural source. In some embodiments, the MSM is a synthetic MSM. In some embodiments, D-Mannose to MSM weight ratio is in the range of 30:1 and 1:2. In some embodiments, D-Mannose to MSM weight ratio is in the range of 10:1 and 1:1. In some embodiments, Phellodendron Extract to MSM weight ratio is in the range of 20:1 and 1:2. In some embodiments, Phellodendron Extract to MSM weight ratio is in the range of 8:1 and 1:1. In some embodiments, Cranberry to MSM weight ratio is in the range of 10:1 and 1:2. In some embodiments, Cranberry to MSM weight ratio is in the range of 4:1 and 1:1.

In some embodiments, MSM weight content is in the range of 1 and 50% from the total weight of the composition. In some embodiments, MSM weight content is in the range of 1 and 30% from the total weight of the composition. In some embodiments, MSM weight content is in the range of 1 and 20% from the total weight of the composition. In some embodiments, MSM weight content is in the range of 1 and 10% from the total weight of the composition. In some embodiments, MSM weight content is in the range of 2 and 8% from the total weight of the composition.

In some embodiments, the composition further comprises Cinnamon. In some embodiments, Cinnamon is a cinnamon bark extract. In some embodiments, D-Mannose to Cinnamon weight ratio is in the range of 40:1 and 1:2. In some embodiments, D-Mannose to Cinnamon weight ratio is in the range of 20:1 and 1:1. In some embodiments, Phellodendron Extract to Cinnamon weight ratio is in the range of 30:1 and 1:2. In some embodiments, Phellodendron Extract to Cinnamon weight ratio is in the range of 15:1 and 1:1. In some embodiments, Cranberry to Cinnamon weight ratio is in the range of 15:1 and 1:2. In some embodiments, Cranberry to Cinnamon weight ratio is in the range of 8:1 and 1:1. In some embodiments, MSM to Cinnamon weight ratio is in the range of 10:1 and 1:8. In some embodiments, MSM to Cinnamon weight ratio is in the range of 6:1 and 1:2.

In some embodiments, Cinnamon weight content is in the range of 1 and 50% from the total weight of the composition. In some embodiments, Cinnamon weight content is in the range of 1 and 40% from the total weight of the composition. In some embodiments, Cinnamon weight content is in the range of 1 and 30% from the total weight of the composition. In some embodiments, Cinnamon weight content is in the range of 1 and 20% from the total weight of the composition. In some embodiments, Cinnamon weight content is in the range of 1 and 10% from the total weight of the composition. In some embodiments, Cinnamon weight content is in the range of 1 and 6% from the total weight of the composition.

In some embodiments, the weight ratio of: D-Mannose to Phellodendron Extract to Cranberry, is in the range of 10:8:1 and 1:1:4 within a composition as described herein. In some embodiments, the weight ratio of: D-Mannose to Phellodendron Extract to Cranberry, is in the range of 4:4:1 and 4:1:4 within a composition as described herein. In some embodiments, the weight ratio of: D-Mannose to Phellodendron Extract to Cranberry, is in the range of 4:4:1 and 2:1:2 within a composition as described herein. In some embodiments, weight ratio of: D-Mannose to Phellodendron Extract to Cranberry is 5:4:2 within a composition as described herein.

In some embodiments, the composition described herein, comprises D-Mannose, Phellodendron Extract and Cranberry. In some embodiments, the composition having an anti-microbial urinary tract infection (UTI) effect. In some embodiments, the composition is inhibiting the attachment of a microorganism to a urinary tract wall. In some embodiments, the composition is inhibiting the attachment of a microorganism to an epithelial cell.

The phrase “Urinary tract infection (UTI)” refers to an infection of the urinary system, which includes the kidneys (the organ that filters the blood to make urine), the ureters (the tubes that take urine from each kidney to the bladder), the bladder (stores urine), or the urethra (the tube that empties urine from the bladder to the outside).

The phrase “microbial urinary tract infection” refers to a case where a pathogenic microorganism invades and colonizes the urinary tract, which may evoke the following symptoms: (a) a burning feeling upon urination; (b) A frequent or intense urge to urinate, even though a small amount of urine is secreted; (c) a pain or pressure in the back or lower abdomen; (d) a cloudy, dark, bloody, or strange-smelling urine; (e) Feeling tired or shaky; (f) a fever or chills.

The term “microorganism” includes a single-celled or multicellular organism, i.e. a bacterium, yeast or fungus.

According to some embodiments of the present invention, there is provided a method of treating or preventing a UTI described herein. In one embodiment, a UTI is a urinary tract microbial infection.

In some embodiments, the present invention further provides a method for treating a subject afflicted with a UTI, comprising the step of administering to the subject a therapeutically effective amount of the composition described herein. In some embodiments, treating a subject afflicted with a UTI, is inhibiting the attachment of a microorganism to an epithelial cell.

In some embodiments, the method further comprises treatment with a UTI antibiotic. In some embodiments, the urinary tract antibiotic is an oral formulation or an injectable formulation. In some embodiments, the oral antibiotic is selected from without being limited thereto, Sulfamethoxazole-trimethoprim, Nitrofurantoin, Fosfomycin, Amoxicillin/clavulanate, cephalosporins, Ceftriaxone, Gentamicin and Tobramycin Ceftriaxone, Gentamicin and Tobramycin.

In some embodiments, the urinary tract antibiotic is administered together with the composition described herein. In some embodiments, the urinary tract antibiotic and the composition described herein are administered separately. In some embodiments, the urinary tract antibiotic is administered prior to the composition described herein. In some embodiments, the urinary tract antibiotic is administered after the composition described herein.

In some embodiments, the composition comprises a urinary tract antibiotic.

The term “Urinary tract antibiotic” refers to an antibiotic drug used to treat a urinary tract infection. Most antibiotics are targeted against the bacteria Escherichia coli, which causing 75-95% off infections in women. The most common oral antibiotics to treat a urinary tract infection are Sulfamethoxazole-trimethoprim, Nitrofurantoin, Fosfomycin and Amoxicillin/clavulanate.

In some embodiments, the present invention further provides a method for reducing the minimal effective dose of a urinary tract antibiotic.

The phrase “minimal effective dose” refers to the minimal dose of a pharmaceutical product exerts a clinically outcome such as reducing the number of infective bacteria in the urinary tract and/or reducing a symptom of UTI as described herein.

In some embodiments, the present invention further provides a method for preventing a urinary tract microbial infection in a subject, comprising the step of administering to the subject a therapeutically effective amount of the composition described herein. In some embodiments, preventing a urinary tract microbial infection in a subject is achieved by a daily supplementation of the composition described herein. In some embodiments, preventing a urinary tract microbial infection is inhibiting the attachment of bacteria to the urinary tract. In some embodiments, preventing a urinary tract microbial infection is inhibiting the attachment of an infection causing bacterium to the urinary tract. In some embodiments, preventing a urinary tract microbial infection is inhibiting the attachment of an infection causing bacteria to an epithelial cell within the urinary tract.

Various embodiments of dosage ranges are contemplated by this invention. The dosage of the composition of the present invention, in some embodiments, is in the range of 0.5-5000 mg/day. In some embodiments, the dosage is in the range of 5-500 mg/day. In some embodiments, the dosage is in the range of 500-2000 mg/day. In some embodiments, the dosage is in the range of 0.1-10 mg/day. In some embodiments, the dosage is in the range of 50-500 mg/day. In some embodiments, the dosage is in the range of 5-4000 mg/day. In some embodiments, the dosage is in the range of 0.5-50 mg/day. In some embodiments, the dosage is in the range of 5-80 mg/day. In some embodiments, the dosage is in the range of 100-1000 mg/day. In some embodiments, the dosage is in the range of 1000-2000 mg/day. In some embodiments, the dosage is in the range of 200-600 mg/day. In some embodiments, the dosage is in the range of 400-1500 mg/day. In some embodiments, the dosage is in a range of 800-1500 mg/day. In some embodiments, the dosage is in the range of 500-2500 mg/day. In some embodiments, the dosage is in a range of 600-1200 mg/day. In some embodiments, the dosage is in the range of 1200-2400 mg/day. In some embodiments, the dosage is in the range of 40-60 mg/day. In some embodiments, the dosage is in a range of 2400-4000 mg/day. In some embodiments, the dosage is in a range of 450-1500 mg/day. In some embodiments, the dosage is in the range of 1500-2500 mg/day. In some embodiments, the dosage is in the range of 5-10 mg/day. In some embodiments, the dosage is in the range of 550-1500 mg/day.

In some embodiments, “dosage” refers to the amount of a composition as described herein. In some embodiments, “dosage” refers to the amount of an active ingredient or the combination of active ingredients of the invention. In another embodiment, “dosage” is not inclusive with respect to excipients. Aqueous solutions, buffers, vehicles, or any other inert substance.

The phrase “active ingredient” refers to the ingredient in a pharmaceutical or nutraceutical formulation that is biologically active. i.e., disclosed herein the following active ingredients: Phellodendron, D-mannose, Cranberry, M S M and Cinnamon, or any combination thereof.

In some embodiments, the dosage is 200 mg/day. In some embodiments, the dosage is 300 mg/day. In some embodiments, the dosage is 400 mg/day. In some embodiments, the dosage is 500 mg/day. In some embodiments, the dosage is 600 mg/day. In some embodiments, the dosage is 700 mg/day. In some embodiments, the dosage is 800 mg/day. In some embodiments, the dosage is 900 mg/day. In some embodiments, the dosage is 1000 mg/day. In some embodiments, the dosage is 1200 mg/day. In some embodiments, the dosage is 1300 mg/day. In some embodiments, the dosage is 1400 mg/day. In some embodiments, the dosage is 1500 mg/day. In some embodiments, the dosage is 1600 mg/day. In some embodiments, the dosage is 1700 mg/day. In some embodiments, the dosage is 1800 mg/day. In some embodiments, the dosage is 1900 mg/day. In some embodiments, the dosage is 2000 mg/day.

In some embodiments, the composition and the method described herein, treat or prevent amicrobial UTI, wherein the infection is a bacterial infection. In some embodiments, “infection” is a yeast infection. In some embodiments, “infection” is a fungal infection.

In some embodiments, the subject is a human. In some embodiments, the subject is a female.

While in the above-disclosed methods, the therapeutic composition may be administered by any convenient means, in one embodiment the composition is administered in a pharmaceutical, a nutraceutical or a nutritional oral form. In another preferred embodiment, however, the therapeutic composition is incorporated into a foodstuff or beverage.

In some embodiments, the composition of the present invention can be provided to the individual per-se. In some embodiments, the composition of the present invention is mixed with a pharmaceutically acceptable carrier.

In some embodiments, the phrases “physiologically acceptable carrier” and “pharmaceutically acceptable carrier” are used interchangeably.

In some embodiments, “excipient” refers to an inert substance added to a and/or nutraceutical composition to further facilitate administration of an active ingredient. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, Pa., latest edition, which is incorporated herein by reference in its entirety.

In some embodiments, oral administration of the composition comprises a unit dosage form comprising tablets, capsules, lozenges, chewable tablets, suspensions, drinks, syrups, nectars, beverages, emulsions and the like. Such unit dosage forms comprise a safe and effective amount of the composition. In some embodiments, the composition is an oral composition. In some embodiments, the oral composition is a capsule. The pharmaceutically-acceptable carriers suitable for the preparation of unit dosage forms for oral administration is well-known in the art. In some embodiments, tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. In one embodiment, glidants such as silicon dioxide can be used to improve flow characteristics of the powder-mixture. In one embodiment, coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. In some embodiments, the selection of carrier components depends on secondary considerations like taste, cost, and shelf stability, which are not critical for the purposes of this invention, and can be readily made by a person skilled in the art.

In some embodiments, the oral dosage form comprises predefined release profile. In one embodiment, the oral dosage form of the present invention comprises an extended release tablets, capsules, lozenges or chewable tablets. In some embodiments, the oral dosage form of the present invention comprises a slow release tablets, capsules, lozenges or chewable tablets. In some embodiments, the oral dosage form of the present invention comprises an immediate release tablets, capsules, lozenges or chewable tablets. In some embodiments, the oral dosage form is formulated according to the desired release profile of the active ingredient as known to one skilled in the art. In another embodiment, the composition is a drink or a beverage comprising a dosage which consists a combination of the active ingredients in a ratio or in an amount as described herein.

In some embodiments, the oral composition comprises liquid solutions, emulsions, suspensions, and the like. In some embodiments, pharmaceutically-acceptable carriers suitable for preparation of such compositions are well known in the art. In some embodiments, liquid oral compositions comprise from about 0.012% to about 0.933% of the active ingredients, or in another embodiment, from about 0.033% to about 0.7%.

In some embodiments, pharmaceutical and/or nutraceutical compositions of this invention comprise solutions or emulsions, which in some embodiments are aqueous solutions or emulsions comprising a safe and effective amount of the composition of the present invention and optionally, other compounds. In some embodiments, the compositions comprise from about 0.01% to about 10.0% w/v or w/w of a combination of active ingredients as described herein.

In some embodiments, and/or nutraceutical compositions of the present invention are manufactured by processes well known in the art, e.g., by means of conventional mixing, dissolving, granulating, dragee-making, levigating, emulsifying, encapsulating, entrapping or lyophilizing processes.

In some embodiments, compositions for use in accordance with the present invention is formulated in conventional manner using one or more physiologically acceptable carriers comprising excipients and auxiliaries, which facilitate processing of the active ingredients into preparations which, can be used pharmaceutically. In some embodiments, formulation is dependent upon the route of administration chosen.

In some embodiments, the composition comprises dietary fibers. In some embodiments, the dietary fiber is inulin, pectin or cellulose.

In some embodiments, the composition comprises preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, compositions include aqueous solutions of the active preparation in water-soluble form. Additionally, suspensions of the active ingredients, in some embodiments, are prepared as appropriate oily or water based suspensions. Suitable lipophilic solvents or vehicles include, in some embodiments, fatty oils such as sesame oil, or synthetic fatty acid esters such as ethyl oleate, triglycerides or liposomes. Aqueous suspensions contain, in some embodiments, substances, which increase the viscosity of the suspension, such as sodium carboxymethyl cellulose, sorbitol or dextran. In another embodiment, the suspension also contains suitable stabilizers or agents which increase the solubility of the active ingredients to allow for the preparation of highly concentrated solutions.

In some embodiments, compositions suitable for use in context of the present invention include compositions wherein the active ingredients are contained in an amount effective to achieve the intended purpose. In some embodiments, a therapeutically effective amount means an amount of active ingredients effective to prevent, alleviate or ameliorate symptoms of disease or prolong the survival of the subject being treated.

In some embodiments, determination of a therapeutically effective amount is well within the capability of those skilled in the art.

Some examples of substances which can serve as nutraceutical or pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerin, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a nutraceutical or a pharmaceutically-acceptable carrier to be used in conjunction with the compound is basically determined by the way the compound is to be administered. If the subject compound is to be injected, in one embodiment, the nutraceutical or pharmaceutically-acceptable carrier is sterile, physiological saline, with a blood-compatible suspending agent, the pH of which has been adjusted to about 7.4.

In addition, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. In another embodiment, peroral liquid compositions also contain one or more components such as sweeteners, flavoring agents and colorants disclosed above.

The compositions also include incorporation of the active ingredient, the compositions of the invention, into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance. Also, comprehended by the invention are particulate compositions coated with polymers (e.g. poloxamers or poloxamines).

In some embodiments, preparation of effective amount or dose can be estimated initially from in vitro assays. In one embodiment, a dose can be formulated in animal models and such information can be used to more accurately determine useful doses in humans.

In some embodiments, toxicity and therapeutic efficacy of the composition described herein can be determined by standard and/or nutraceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

In some embodiments, depending on the severity and responsiveness of the condition to be treated, dosing can be of a single or a plurality of administrations, with course of treatment lasting from several days to several weeks or until cure is effected or diminution of the disease state is achieved.

In some embodiments, the amount of a composition to be administered will, of course, be dependent on the subject being treated, the severity of the affliction, the manner of administration, the judgment of the prescribing physician, etc.

In some embodiments, compositions including the preparation of the present invention formulated in a compatible and/or nutraceutical carrier are also prepared, placed in an appropriate container, and labeled for treatment of an indicated condition.

In some embodiments, compositions of the present invention are presented in a pack or dispenser device, such as an FDA approved kit, which contain one or more unit dosage forms containing the composition. In one embodiment, the pack, for example, comprise metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of and/or nutraceutical, which notice is reflective of approval by the agency of the form of the compositions or human or veterinary administration. Such notice, in one embodiment, is labeling approved by the U.S. Food and Drug Administration for prescription drugs or of an approved product insert.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Example 1: Anti-UTI Exemplary Formulation

The following anti-UTI composition was used according to the invention (table 1)

| Botanical name | Weight | Brand name |
|---|---|---|
| D-Mannose | 250 mg | D-Mannose |
| Phellodendron chinense extract | 190 mg | Phellodendron |
| | | chinense EX |
| | 100 mg | CRANMAX ® |
| Methylsulfonymethane (MSM) | 30 mg | methylsulfonymethane |
| | | (MSM) |
| Cinnamon bark extract | 20 mg | Cinnamon bark Ex |
| Magnesium (Citrate tribasic) | 10 mg Mg Citratet | Magnesium Citrate |
| hydrate | (=1.5 mg Mg) | |
| Inulin dietary fibers | 20 mg | |
| Vegetarian capsule 00 | 120 mg | |
| Vegetarian Capsule 00 | | |

### Example 2: Human Red Blood Cell (HRBC) Agglutination Assay

Materials and Methods

A whole blood (type 0 positive) was acquired from a volunteer and stored with EDTA at 4° C. Before the agglutination experiment, the red blood cells were isolated by centrifugation at 800 rpm for 15 minutes (in order to remove the platelets and plasma). The resulting red blood cells were suspended in phosphate buffered (PBS, pH 7.4) and washed three times to remove EDTA and other blood components, after each wash the supernatant was removed and the remaining cells were resuspended in PBS. Finally, RBC were diluted in 10 ml of PBS to create 1% RBC suspension that was stored at 4° C. until use.

Standard E. coli strains (NCTC 4169 and NCTC 9775) were grown on TSA plates for 24 h at 37° C. Colony was collected with sterile culturing needle and cells suspended in PBS and washed three times with PBS, after each wash the supernatant was removed. The cells were resuspended in PBS at final concentration of 10⁹ cells/ml and stored at 4° C. for an hour.

The experiments were conducted in round bottom 96-well plate, with final volume 200 μl, made of 98 μl of the 1% RBC suspension, 2 μl extract (or PBS) and 100 μl bacterial suspension or PBS (as control). Extract were prepared in advance at different concentrations in PBS (10 mg/ml, 3 mg/ml, 1 mg/ml, 50 μg/ml and 1 μg/ml). Were applicable the RBC suspension and the tested extract were pre-mixed in order to allow binding (for 10 minutes) and then bacteria was added. The plate was incubated at 4° C. and observed after 30 minutes, 60 minutes and 120 minutes.

Results

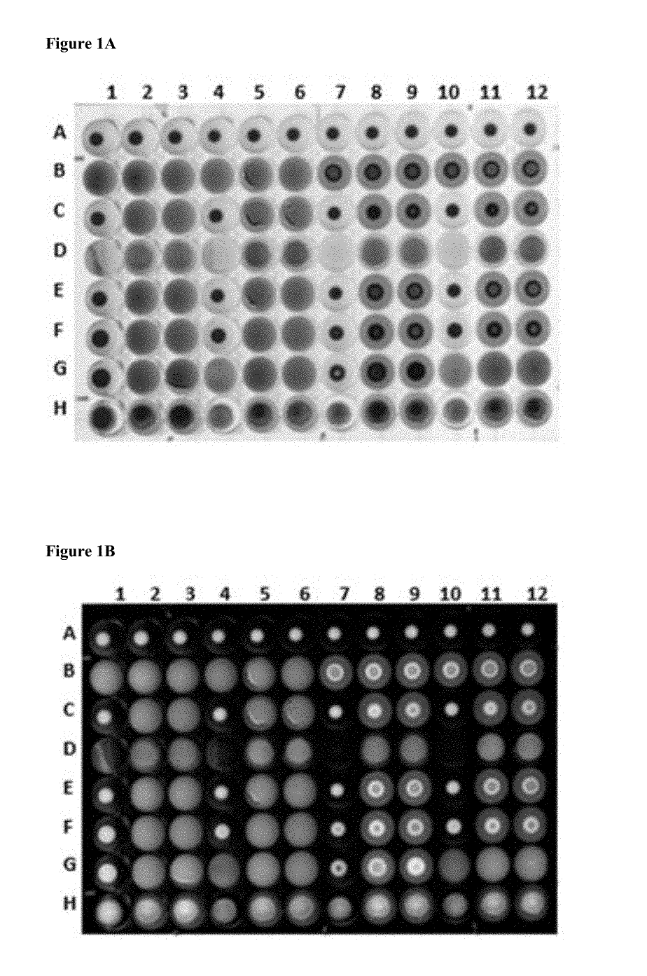

Tested controls: (a) wells without bacteria showed no agglutination and the red blood cells sank to the bottom of the well (FIG. 1 (A or B), Raw A) while (b) “positive” control (RBC+Bacteria) showed strong agglutination (different agglutination patterns for different bacterial strains) (FIG. 1 (A or B), Raw B).

Strain 9775 was D-mannose sensitive, while strain 4169 was not D-mannose sensitive (FIG. 1 (A or B), Raw C).

Through the combinations and various concentrations examined, it was shown that substances that induced agglutination prevention/inhibition are D-mannose (5-10 mg/ml), Phellodendron (10 mg/ml) and cranberries (5 mg/ml) extracts.

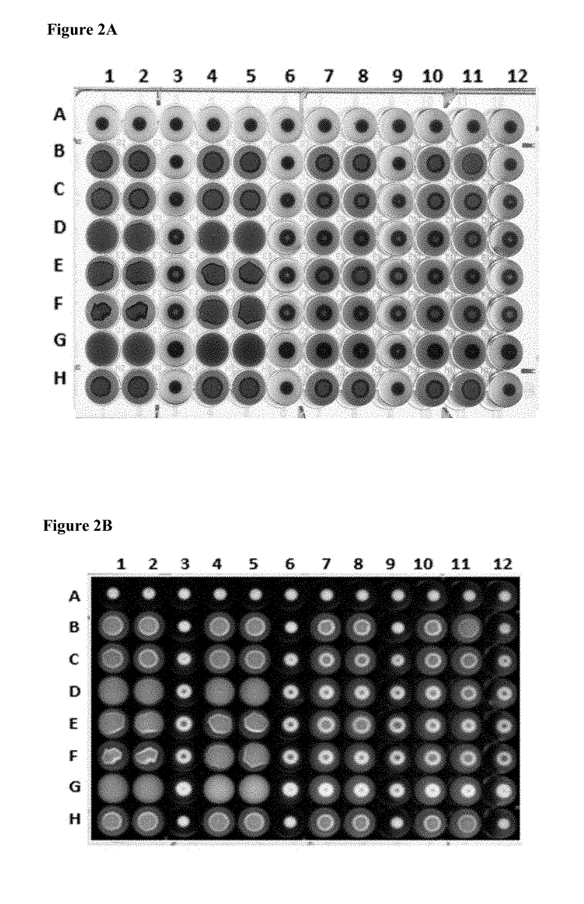

The composition consisting D-mannose, Phellodendron and Cranberry, with the following ingredients weight ratio: D-mannose to Phellodendron to Cranberry of 2.5:1.9:1 was found as the most effective in preventing E. coli NCTC 9775 induced agglutination of RBC (FIG. 2 (A or B)).

1. A composition comprising D-Mannose and Phellodendron Extract, wherein said D-Mannose weight is from 3.5% to 85% of the total weight of said composition.

2. The composition of claim 1, wherein said composition further comprises a Cranberry.

3. The composition of claim 1, wherein said composition further comprises Methylsulfonylmethane (MSM).

4. The composition of claim 1, wherein said composition further comprises Cinnamon.

5. The composition of claim 2, wherein weight ratio of D-Mannose to Phellodendron Extract to Cranberry is from 4:4:1 to 4:1:2.

6. The composition of claim 1, wherein said composition is an oral composition.

7. The composition of claim 6, wherein said oral composition is selected from the group consisting tablet, capsule, solution, powder, suspension, syrup or beverage.

8. A method of treating a subject afflicted with a urinary tract microbial infection, comprising the step of administering to said subject a therapeutically effective amount of a composition of claim 1, thereby treating a subject afflicted with a urinary tract microbial infection.

9. The method of claim 8, wherein said treating a subject afflicted with a urinary tract microbial infection is inhibiting the attachment of a microorganism to an epithelial cell.

10. The method of claim 8, further comprising administering to said subject a urinary tract antibiotic.

11. A method of preventing a urinary tract microbial infection in a subject, comprising the step of administering to a subject a therapeutically effective amount of a composition of claim 1, thereby preventing a urinary tract microbial infection in a subject.

12. The method of claim 11, wherein said preventing comprises daily supplementation of said composition.

13. A method for reducing a minimal effective dose of a urinary tract antibiotic in treating a subject afflicted with a urinary tract microbial infection, comprising the step of administering to said subject a therapeutically effective amount of a composition of claim 1 along with said antibiotic, thereby reducing the minimal effective dose of a urinary tract antibiotic in treating a subject afflicted with a urinary tract microbial infection.

14. The method of claim 8 wherein said urinary tract microbial infection is bacterial infection.

15. The method of claim 8, wherein said urinary tract microbial infection is yeast infection.

16. The method of claim 8, wherein said urinary tract microbial infection is fungal infection.

17. The method of claim 8, wherein said subject is a female.
